(19) United States
(12) Patent Application Publication
(10) Pub. No.: US 2018/0368744 A1
(43) Pub. Date: Dec. 27, 2018

(54) URINE CATHETER PH SENSOR

(71) Applicant: INTERNATIONAL BUSINESS MACHINES CORPORATION, Armonk, NY (US)

(72) Inventors: AMOS CAHAN, DOBBS FERRY, NY (US); SUFI ZAFAR, BRIARCLIFF MANOR, NY (US)

(21) Appl. No.: 15/632,420

(22) Filed: Jun. 26, 2017

(51) Int. Cl. A61B 5/145; A61B 5/00; A61B 5/20

(52) U.S. Cl. CPC A61B 5/14539; A61B 5/6852; A61M 2205/3324; A61B 5/14507; A61B 5/202

(57) ABSTRACT

Embodiments of the present invention are directed to a system for sensing urine pH. A non-limiting example of the system includes a urinary catheter tube for insertion into a bladder of a patient, wherein the urine catheter tube includes an inner cavity. The system also includes a collection vessel connected to the urinary catheter tube. The system also includes a FET-based pH sensing device including a pH sensing surface and a reference electrode, wherein the pH sensing surface and the reference electrode have a surface exposed to the inner cavity. Such embodiments can advantageously provide a real-time, sensitive measurement of urine for timely detection and monitoring of the physiological condition of a subject with a miniaturized FET-based pH sensor.

### BACKGROUND

The present invention generally relates to medical devices and methods, and more specifically to urine catheter pH sensors and related pH sensing methodologies.

In the body of a healthy subject, pH can be closely regulated and maintained within a narrow range through a broad set of physiological mechanisms. Changes in the pH of body fluids can indicate a change in a subject's physiological condition. Such pH changes could have diagnostic and therapeutic implications. Detecting pH in urine, therefore, can aid in diagnosing and monitoring patient health.

Although point measurement of urine pH can be simple, obtaining a real-time, accurate pH of a sample poses several challenges. For example, a conventional method of rapidly determining pH involves can be performed at a patient's bedside with a pH sensitive stick with a color indicator. However, such indicators can lack sufficient sensitivity to respond to urine pH changes associated with physiological changes. Although greater sensitivity of a urine sample pH can be achieved by submitting the sample to a laboratory, such methods can lack timeliness and are not amenable to real-time patient monitoring. Moreover, timeliness in urine pH measurement can be important because urine pH can change over time due to bacteria present in the urine. Thus, methods of pH analysis of urine that involve delays can lead to inaccurate information and are not consistently representative of a physiological status of a patient. There remains a need for timely, sensitive urine pH measurement for diagnostic and therapeutic treatment of a patient.

### SUMMARY

Embodiments of the present invention are directed to a system for sensing urine pH. A non-limiting example of the system includes a urinary catheter tube for insertion into a bladder of a patient, wherein the urine catheter tube includes an inner cavity. The system also includes a collection vessel communicatively coupled to the urinary catheter tube. The system also includes a transistor-based pH sensing device including a pH sensing surface and a reference electrode, wherein the pH sensing surface and the reference electrode have a surface exposed to the inner cavity. Such embodiments of the invention can advantageously provide a real-time, sensitive measurement of urine for timely detection and monitoring of the physiological condition of a subject with a miniaturized transistor-based pH sensor.

Embodiments of the present invention are directed to a system for sensing urine pH. A non-limiting example of the system includes a urinary catheter tube for insertion into a bladder of a patient, wherein the urine catheter tube includes an inner cavity. The system also includes a collection vessel communicatively coupled to the urinary catheter tube. The system also includes a pH sensing device including a reference electrode and a pH sensing surface connected to the base of a BJT device. The BJT device further includes a collector and an emitter. The pH sensing surface and the reference electrode have a surface exposed to the inner cavity. Such embodiments of the invention can advantageously provide a real-time, sensitive measurement of urine for timely detection and monitoring of the physiological condition of a subject with a miniaturized BJT-based pH sensor.

Embodiments of the present invention are directed to a method for sensing urinary pH. A non-limiting example of the method includes receiving a signal from a pH sensor in a catheter tube channel applied to fresh urine in the channel. The pH sensor includes a sensing surface, a collector and a reference electrode. The method also includes applying zero voltage to the collector. The method also includes applying zero voltage to the reference electrode. The method also includes applying a voltage to the emitter. The method also includes measuring a collector current from the collector. Such embodiments of the invention can provide highly sensitive pH measurements for determination of urine pH in a catheter system.

Embodiments of the present invention are directed to a method for sensing urinary pH. A non-limiting example of the method includes placing a pH sensing apparatus in contact with urine of a patient. The apparatus can include a urinary catheter tube for insertion into a bladder of a patient. The urine catheter tube includes an inner cavity. The system can also include a collection vessel connected to the urinary catheter tube. The system can also include a transistor-based pH sensing device including a pH sensing surface and a reference electrode. The pH sensing surface and the reference electrode have a surface exposed to the inner cavity. Such embodiments of the invention can provide highly sensitive pH measurements for determination of urine pH in a catheter system.

Embodiments of the present invention are directed to a method for sensing urinary pH. A non-limiting example of the method includes placing a pH sensing apparatus in contact with urine of a patient. The apparatus includes a urinary catheter tube for insertion into a bladder of a patient. The urine catheter tube includes an inner cavity. The apparatus also includes a collection vessel connected to the urinary catheter tube. The apparatus also includes a pH sensing device including a reference electrode and a pH sensing surface connected to the base of a BJT device. The BJT device further includes a collector and an emitter. The pH sensing surface and the reference electrode have a surface exposed to the inner cavity. The method also includes determining a real-time urine pH of the patient. Such embodiments of the invention can advantageously provide a real-time, sensitive measurement of urine for timely detection and monitoring of the physiological condition of a subject with a miniaturized BJT-based pH sensor.

Additional technical features and benefits are realized through the techniques of the present invention. Embodiments and aspects of the invention are described in detail herein and are considered a part of the claimed subject matter. For a better understanding, refer to the detailed description and to the drawings.

### BRIEF DESCRIPTION OF THE DRAWINGS

The specifics of the exclusive rights described herein are particularly pointed out and distinctly claimed in the claims at the conclusion of the specification. The foregoing and other features and advantages of the embodiments of the invention are apparent from the following detailed description taken in conjunction with the accompanying drawings in which:

FIG. 1 depicts a block diagram illustrating one example of a processing system according to one or more embodiments of the present invention.

FIG. 2 depicts an exemplary system according to one or more embodiments of the present invention.

FIG. 3 depicts an exemplary system according to one or more embodiments of the present invention.

FIG. 4 depicts an exemplary system according to one or more embodiments of the present invention.

FIG. 5 is a chart depicting solution voltage versus drain current for an exemplary system according to one or more embodiments of the present invention.

FIG. 6 depicts an exemplary system according to one or more embodiments of the present invention.

FIG. 7 depicts an exemplary system according to one or more embodiments of the present invention.

FIG. 8 depicts a flow diagram of an exemplary method according to one or more embodiments of the present invention.

The diagrams depicted herein are illustrative. There can be many variations to the diagram or the operations described therein without departing from the spirit of the invention. For instance, the actions can be performed in a differing order or actions can be added, deleted or modified. Also, the term “coupled” and variations thereof describes having a communications path between two elements and does not imply a direct connection between the elements with no intervening elements/connections between them. All of these variations are considered a part of the specification.

In the accompanying figures and following detailed description of the described embodiments, the various elements illustrated in the figures are provided with two or three digit reference numbers. With minor exceptions, the leftmost digit(s) of each reference number correspond to the figure in which its element is first illustrated.

### DETAILED DESCRIPTION

Various embodiments of the invention are described herein with reference to the related drawings. Alternative embodiments of the invention can be devised without departing from the scope of this invention. Various connections and positional relationships (e.g., over, below, adjacent, etc.) are set forth between elements in the following description and in the drawings. These connections and/or positional relationships, unless specified otherwise, can be direct or indirect, and the present invention is not intended to be limiting in this respect. Accordingly, a coupling of entities can refer to either a direct or an indirect coupling, and a positional relationship between entities can be a direct or indirect positional relationship. Moreover, the various tasks and process steps described herein can be incorporated into a more comprehensive procedure or process having additional steps or functionality not described in detail herein.

The following definitions and abbreviations are to be used for the interpretation of the claims and the specification. As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance or illustration.” Any embodiment or design described herein as “exemplary” is not necessarily to be construed as preferred or advantageous over other embodiments or designs. The terms “at least one” and “one or more” can include any integer number greater than or equal to one, i.e. one, two, three, four, etc. The terms “a plurality” can include any integer number greater than or equal to two, i.e. two, three, four, five, etc. The term “connection” can include both an indirect “connection” and a direct “connection.”

The terms “about,” “substantially,” “approximately,” and variations thereof, are intended to include the degree of error associated with measurement of the particular quantity based upon the equipment available at the time of filing the application. For example, “about” can include a range of ±8% or 5%, or 2% of a given value.

For the sake of brevity, conventional techniques related to making and using aspects of the invention may or may not be described in detail herein. In particular, various aspects of computing systems and specific computer programs to implement the various technical features described herein are well known. Accordingly, in the interest of brevity, many conventional implementation details are only mentioned briefly herein or are omitted entirely without providing the well-known system and/or process details.

Additionally, conventional techniques related to semiconductor device and integrated circuit (IC) fabrication may or may not be described in detail herein. Moreover, the various tasks and process steps described herein can be incorporated into a more comprehensive procedure or process having additional steps or functionality not described in detail herein. In particular, various steps in the manufacture of semiconductor devices and semiconductor-based ICs are well known and so, in the interest of brevity, many conventional steps will only be mentioned briefly herein or will be omitted entirely without providing the well-known process details.

Spatially relative terms, e.g., “beneath,” “below,” “lower,” “above,” “upper,” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device can be otherwise oriented (rotated 90 degrees or at other orientations) and the spatially relative descriptors used herein interpreted accordingly.

Turning now to an overview of technologies that are more specifically relevant to aspects of the invention, urine pH can be an important clinical measure. Abnormal urine pH can, for example, result from a kidney malfunction or a systemic acid-base disorder resulting from a variety of causes. For instance, intoxication, respiratory problems, urinary tract infections, impaired perfusion to tissues, and metabolic disorders such as diabetes can all lead to an abnormal urine pH. In some cases, causing an increase in urine pH through administration of medications can be part of a medical treatment regime. Thus, accurate and timely pH measurements of a subject's urine can provide powerful information in the course of medical diagnosis and treatment.

While a pH measurement can, in some cases, be simply performed, timely and accurate pH monitoring of the urine of an individual can pose several challenges. For example, although bedside pH monitoring with, for instance, a litmus test cannot account for minute to minute pH changes in urine pH and can lack sensitivity. Although increased accuracy and sensitivity of pH measurement could result from sending a urine sample to a laboratory for pH measurement, such methods can nonetheless fail to accurately represent a physiological state. Changes in urine composition, for instance due to microorganism proliferation in the urine over time, could cause a change in the pH such that it no longer reflects the pH of urine within the body. In addition, methods of monitoring pH that result upon collecting urine over time within a urine collection chamber cannot account for minute to minute changes in urine to the extent they do not monitor the urine before it reaches the urine container and, furthermore, because the urine pH can be altered by already collected urine.

There remains a need to provide timely and accurate monitoring of urine pH. Moreover, there remains a need to provide continuous or intermittent real-time monitoring of urine pH before or immediately after the urine leaves a patient's body and before it is mixed with already collected urine.

Turning now to an overview of the aspects of the invention, one or more embodiments of the invention address the above-described shortcomings of conventional methods by providing a catheter system including a sensitive pH sensor for bedside pH monitoring. Embodiments of the invention include a pH sensor within the catheter system, such as within a catheter tube, to provide pH measurements in real-time. For instance, by providing a pH sensor within the catheter tube, pH readings can be instantaneous and sensitive, avoiding the need for laboratory testing and avoiding mixing with prior urine samples, for example, in the collection vessel.

Timely pH measurements can provide medical professionals with robust information concerning patient health without artifacts that would result from bacterial propagation in urine samples that have been allowed to sit for extended periods of time before testing.

The above-described aspects of the invention address the shortcomings of conventional methods by including miniature scale pH sensors embedded in or connected to urinary catheters or other drains used to clear urine from the body of a patient. As used herein, “urinary catheter” is understood to include drains used to clear urine from the body through insertion into the body and having an outlet external to the body. In some embodiments of the invention, field effect transistor (FET)-based pH sensors are included within a urinary catheter system. In some embodiments of the invention, bipolar junction transistor (BJT)-based pH sensors are included within a urinary catheter system.

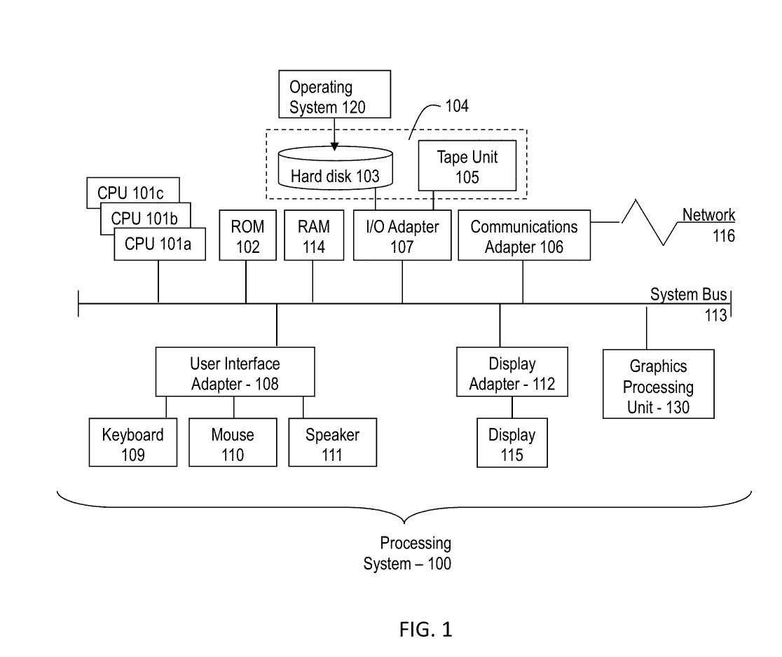

Referring to FIG. 1, there is shown an embodiment of a processing system 100 for implementing the teachings herein. In this embodiment, the system 100 has one or more central processing units (processors) 101a, 101b, 101c, etc. (collectively or generically referred to as processor(s) 101). In one embodiment, each processor 101 can include a reduced instruction set computer (RISC) microprocessor. Processors 101 are coupled to system memory 114 and various other components via a system bus 113. Read only memory (ROM) 102 is coupled to the system bus 113 and can include a basic input/output system (BIOS), which controls certain basic functions of system 100.

FIG. 1 further depicts an input/output (I/O) adapter 107 and a network adapter 106 coupled to the system bus 113. I/O adapter 107 can be a small computer system interface (SCSI) adapter that communicates with a hard disk 103 and/or tape storage drive 105 or any other similar component. I/O adapter 107, hard disk 103, and tape storage device 105 are collectively referred to herein as mass storage 104. Operating system 120 for execution on the processing system 100 can be stored in mass storage 104. A network adapter 106 interconnects bus 113 with an outside network 116 enabling data processing system 100 to communicate with other such systems. A screen (e.g., a display monitor) 115 is connected to system bus 113 by display adaptor 112, which can include a graphics adapter to improve the performance of graphics intensive applications and a video controller. In one embodiment, adapters 107, 106, and 112 can be connected to one or more I/O busses that are connected to system bus 113 via an intermediate bus bridge (not shown). Suitable I/O buses for connecting peripheral devices such as hard disk controllers, network adapters, and graphics adapters typically include common protocols, such as the Peripheral Component Interconnect (PCI). Additional input/output devices are shown as connected to system bus 113 via user interface adapter 108 and display adapter 112. A keyboard 109, mouse 110, and speaker 111 all interconnected to bus 113 via user interface adapter 108, which can include, for example, a Super I/O chip integrating multiple device adapters into a single integrated circuit.

In exemplary embodiments of the invention, the processing system 100 includes a graphics processing unit 130. Graphics processing unit 130 is a specialized electronic circuit designed to manipulate and alter memory to accelerate the creation of images in a frame buffer intended for output to a display. In general, graphics processing unit 130 is very efficient at manipulating computer graphics and image processing, and has a highly parallel structure that makes it more effective than general-purpose CPUs for algorithms where processing of large blocks of data is done in parallel.

Thus, as configured in FIG. 1, the system 100 includes processing capability in the form of processors 101, storage capability including system memory 114 and mass storage 104, input means such as keyboard 109 and mouse 110, and output capability including speaker 111 and display 115. In one embodiment, a portion of system memory 114 and mass storage 104 collectively store an operating system such as the AIX® operating system from IBM Corporation to coordinate the functions of the various components shown in FIG. 1.

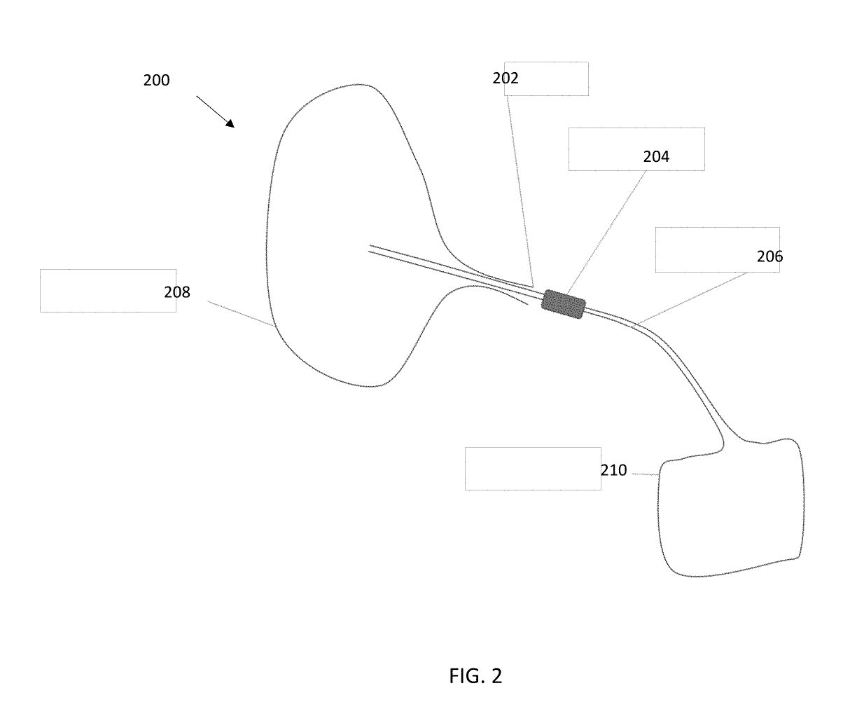

Turning now to a more detailed description of aspects of the present invention, FIG. 2 depicts a urine pH sensing system according to embodiments of the invention. As is shown in FIG. 2, the system 200 includes a urinary catheter tube 206 and a pH sensing device 204. In some embodiments of the invention, the urinary catheter tube 206 includes a collecting vessel 210 at a distal end of the system. The urinary catheter 206, as is illustrated, can be inserted through a urethra 202 into a urinary bladder 208. The pH sensing device 204 can be attached to or integrated with the urinary catheter tube 206, such that urine drained by the urinary catheter tube 206 can come into contact with the pH sensing device 204 on its way to the collecting vessel 210. Thus, such systems can advantageously provide a timely pH measurement of urine before it leaves the body and before it can be mixed with prior urine samples.

In some embodiments of the invention (not shown in FIG. 2), an optional additional pH sensor can be included within the collecting vessel 210 such that it can be placed in contact with urine in the collecting vessel 210. By measuring the pH in the collecting vessel 210 and comparing it to pH measured by a sensor located within the catheter further physiological information, other than urine pH can be determined. For example, by measuring the change of pH over time in the collecting vessel 210 and comparing it to the pH measured by a pH sensor 204 included on or within the urinary catheter 206, the number of bacteria present within the volume of urine can be inferred.

In some embodiments of the invention, the pH sensing device 204 is integrated within the urinary catheter tube 206. In some embodiments of the invention, the pH sensing device 204 is detachable or includes a detachable component. For example, the pH sensing device 204 can include a detachable pH sensor or detachable wires and/or can communicate wirelessly to an external device, such as a computer or smart device.

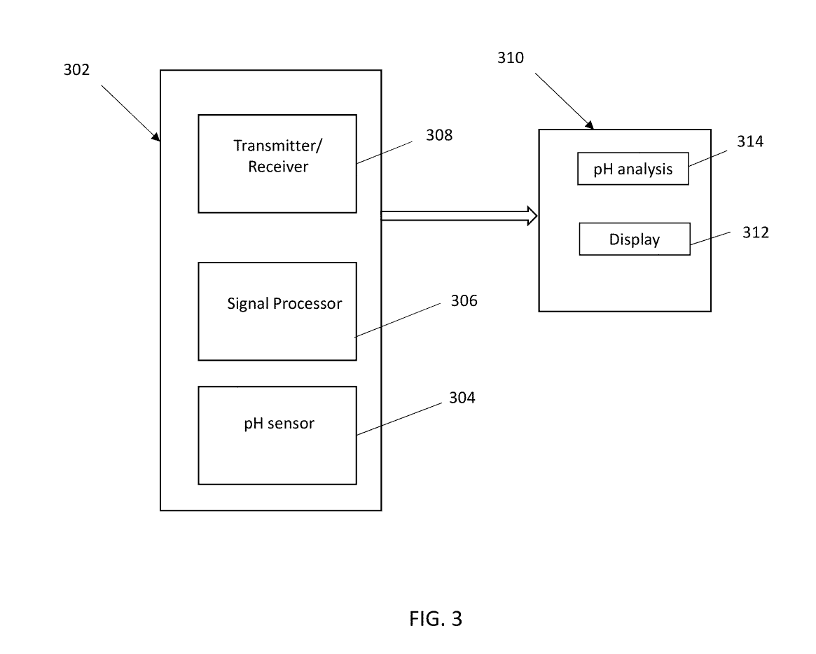

FIG. 3 depicts an exemplary system 300 for measuring the pH of urine according to some embodiments of the invention. The system 300 can include a pH sensor 306. The pH sensor 306 can include, for example, a FET-based pH sensor or a BJT-based pH sensor. The system 300 can also include a signal processor 304 in communication with the pH sensor 306. The signal processor 304 can optionally be connected to the pH sensor 306 via an amplifier (not shown in FIG. 3). In some embodiments of the invention can be generated by the pH sensor 306 and processed by the signal processor 304 and transmitted to an external device 310. The external device 310 can optionally include a pH analysis module 314, for instance for further analysis and recording, and a user interface, such as a display 312.

In some embodiments of the invention, the pH sensor 306 is a disposable sensor that is embedded within the catheter lumen or catheter wall, such that at least a portion of the sensor can come into contact with flowing urine. In some embodiments of the invention, a control unit including a power source, a microprocessor, and a wireless transmitter/receiver can be connected to the pH sensor 306 wirelessly or with a wire, such as a detachable wire. In some embodiments of the invention, a reusable control unit is included. A reusable control unit can be positioned in proximity to the pH sensor 306 such that it maintains communication with the pH sensor. In some embodiments of the invention a control unit is secured to an external (outside of the body when in use) aspect of the catheter.

In some embodiments of the invention, a system includes one or more FET-based pH sensors. The FET-based pH sensors can be provided individually or within an array.

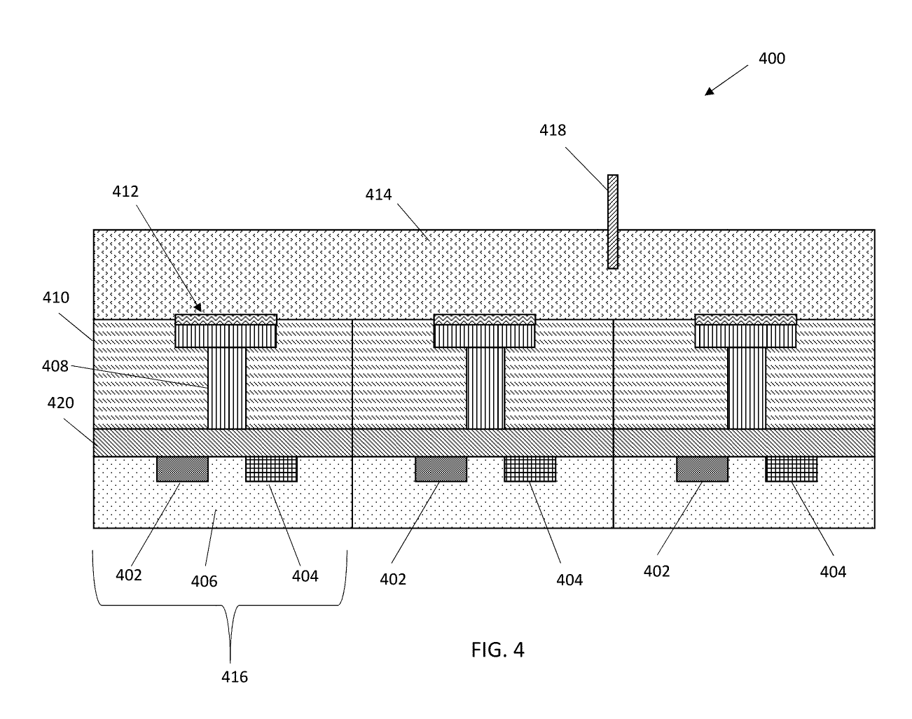

FIG. 4 illustrates an exemplary array of FET-based pH sensors 400 according to one or more embodiments of the present invention. The array 400 includes a plurality of FET-based pH sensors 416, which can each include a FET silicon substrate 406 and a source 402 and drain 404. FET Silicon substrate 406 can include silicon or doped silicon, for example the substrate 406 can include a silicon-on-insulator wafer (SOI) with lightly doped p-type silicon. The FET-pH sensor 416 can include an oxide layer 410. The FET-pH sensor 416 includes a gate dielectric 420 atop the FET silicon substrate 406. The FET-based pH sensor array 400 includes a reference electrode 418. The reference electrode 418 can include, for example, silver chloride. The reference electrode 418 A gate 408, including a pH sensing surface 412, can be embedded within or on top of the oxide layer 410.

Each of the pH sensing surface 412 and the reference electrode 418 can have surfaces externally accessible to the FET-based pH sensor 416 such that they can be placed into contact with urine 414, for example in the channel of a urinary catheter tube or in a collecting vessel. FIG. 4 depicts an embodiment in which urine 414 is placed in contact with the pH sensing surface 412 and reference electrode 418.

Source 402, and drain 404 can be composed of materials conventionally used for such components in FET-devices and can be formed by conventional methods. Source 402 and drain 404 are formed on opposing sides of the gate 608. For example, source 402 and drain 404 can be formed with an epitaxial growth process to deposit a crystalline layer onto the FET substrate 406. The epitaxial silicon, silicon germanium, and/or carbon doped silicon (Si:C) can be doped during deposition by adding a dopant or impurity to form a silicide. The epitaxial source/drain can be doped with an n-type dopant or a p-type dopant, which depends on the type of transistor. In some embodiments of the invention, the source 402 and drain 404 include heavily boron doped source and drain regions. Alternatively, the source/drain 402/404 can be formed by incorporating dopants into the substrate 406.

Oxide layer 410 can be formed over the source 402 and drain 404 and gate dielectric 420 and around the gate 408. The oxide layer 410 can include, for example, a low-k dielectric oxide. In some embodiments of the invention, oxide layer 410 includes tetra-ethyl orthosilicate (TEOS) oxide.

Gate 408 and pH sensing surface 412 can be the same material or different materials and can include any insulating material that is sensitive to pH. In some embodiments of the invention, gate 408 and pH sensing surface 412 are the same material. The pH sensing surface 412 includes a pH sensitive material. In some embodiments of the invention, gate and/or pH sensing surface include hafnium dioxide (HfO₂), aluminum oxide (Al₂O₃), vanadium oxide (V₂O₅), titanium oxide (TiO₂), tungsten oxides, titanium nitride (TiN) or combinations thereof. In some embodiments of the invention, the pH sensing surface 412 (the external surface of the gate) determines a local pH of urine. In some embodiments of the invention, pH sensing surface 412 is composed of HfO₂or TiN.

The pH sensing 412 surface can have any shape, including for instance the shape of a needle. The sensing surface can have a length or diameter of about 5 to about 15 micrometers (μm), for example from about 5 to about 10 μm or from about 5 to about 8 μm.

Sensing of pH with a FET-based pH sensor can be performed in accordance with known methods. In operation, according to some embodiments of the invention, the sensing signal is a drain current I_{D}. Measurements can be made, for example, by setting the reference electrode voltage equal to a gate voltage, setting the drain to a small voltage (e.g., |30 mV|) and setting the source voltage to 0 V. The silicon substrate can be set to 0 V at the back side. A device including a FET-based pH sensor can be applied to a solution, including to urine or a reference or standardized solution for example, such that a sensing surface and reference electrode are exposed to the fluid. Measurements of drain current can be taken and used to determine local pH.

In some embodiments of the invention, an apparatus including a FET-based pH sensor can be calibrated to determine sensing signal dependence on voltage and pH. After calibration, drain current can be measured at a fixed voltage and pH calculated therefrom.

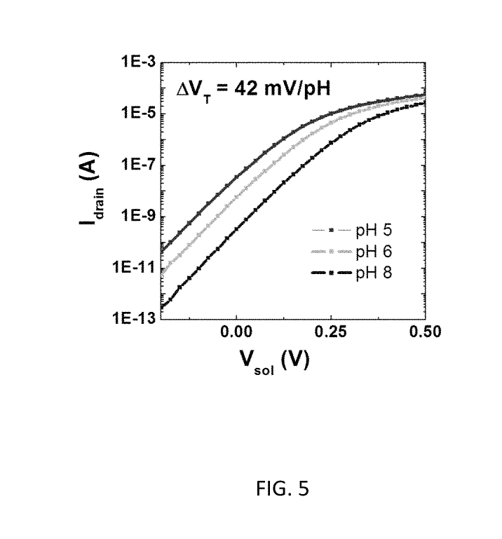

FIG. 5 is a chart depicting drain current (I_{D}) versus gate voltage V_{SOL.} of an exemplary FET-based pH sensor for use in embodiments of the present invention. FIG. 5 demonstrates sensing signal (I_{D}) dependence on gate voltage and pH. Buffered solutions, such as phosphate buffer of 100 mM concentration, having known pH values of 5, 6, and 8 can each be applied to a FET-based pH sensor, such as a FET-based pH sensor for use in embodiments of the present invention. I_{D} can be measured and plotted against the gate voltage V_{SOL.}. FIG. 5 illustrates a FET-based pH sensor with a voltage per pH unit of 42 mV.

In some embodiments of the invention, calibration results are used to determine a pH of urine. For example, a fixed applied voltage can be applied to a system having one or more FET-based pH sensors or an array of FET-based pH sensors and a sensing signal (I_{D}) can be measured in real-time. From the sensing signal, pH can readily be calculated with the calibration results.

In some embodiments of the invention, a system includes one or more BJT-based pH sensors. The BJT-based pH sensors can be provided individually or within an array.

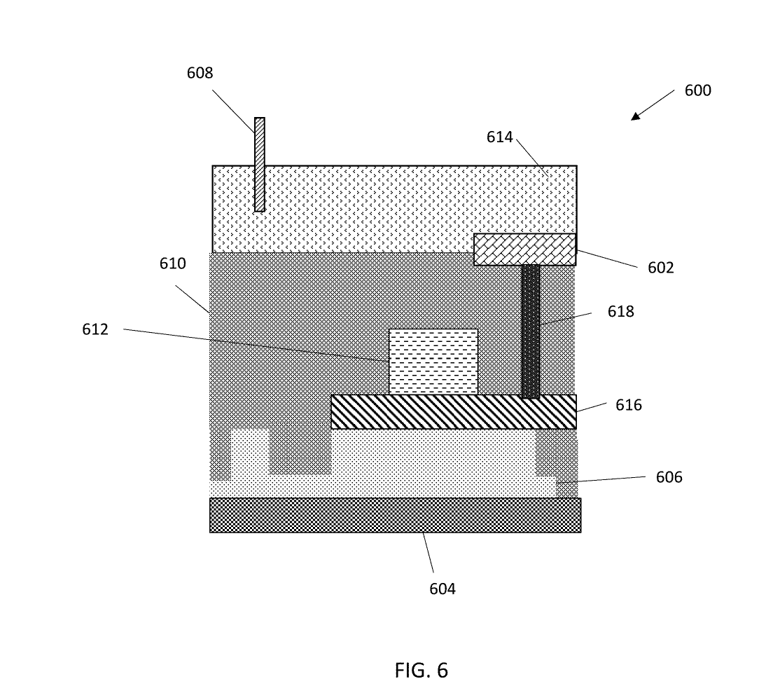

FIG. 6 illustrates an exemplary BJT-based pH sensor 600 for use in a urine catheter system according to one or more embodiments of the present invention. BJT-pH sensor 600 includes a silicon substrate 604 and a collector 606 positioned on the silicon substrate 604. The BJT-pH sensor 600 also includes a base 616 formed on the collector 606. An emitter 612 can be formed on the base 616.

The BJT-pH sensor 600 can be an NPN type BJT or a PNP type BJT device. The selection of materials and dopant polarity can vary depending on whether the BJT-pH sensor is an NPN type or PNP type. For example, an NPN BJT can include a heavily doped n-type emitter 616, a p-type doped base 616, and a p-type doped collector 606. In some embodiments of the invention, the BJT-pH sensor 600 is a PNP type including, for instance, a heavily doped p-type emitter 616, an n-type doped base 616, and an n-type doped collector 606.

Silicon substrate 604 can include silicon or doped silicon. For example, the substrate 604 can include undoped silicon, p-type doped silicon or n-type doped silicon.

Collector 606 can include, for example, silicon, including doped or heavily doped silicon (i.e., more heavily doped than the substrate 604, which can be doped or undoped). The dopant polarity can be opposite to that of the substrate 604. For example, if the substrate 604 includes p-type doped silicon, the collector can include n-type heavily doped silicon. In some embodiments of the invention, collector 406 includes n-type heavily doped gallium arsenide (GaAs).

A base 616 can be formed on the collector 606. Base 616 can include, for instance, a doped silicon, such as silicon germanium (SiGe). In some embodiments of the invention, the silicon germanium is doped, or heavily doped (i.e., more heavily doped than the substrate 604). The dopant polarity can be opposite to that of the collector 606. For example, if the collector 606 includes n-type doped or heavily doped silicon, the base 616 can include p-type doped or heavily doped silicon germanium.

An emitter 612 can be formed on the base 616 and can include, for instance, silicon, polysilicon, or gallium arsenide. Emitter 612 can include polysilicon that is very heavily doped (i.e., doped more heavily than the collector 606 or the base 616).

As is further illustrated in FIG. 6, in one or more embodiments of the present invention BJT-pH sensor 600 includes a reference electrode 608 and a sensing surface 602. The reference electrode 608 can include, for example, a silver chloride reference electrode. The sensing surface 602 and reference electrode 608 can have surfaces externally accessible to the BJT-pH sensor such that they can be placed into contact with fluid, such as urine 614 or saline or buffered solution. In some embodiments of the invention, the sensing surface(s) 602 are accessible to urine when the BJT-pH sensor is included within a catheter system according to one or more embodiments of the present invention. Base 616 can be electrically connected to the sensing surface 602 via a metal line 618. Metal line 618 can be a conductive metal wire, such as a tungsten wire.

The sensing surface 602 is positioned on or embedded within an oxide layer 610. The sensing surface 602 and reference electrode 608 each have an accessible surface for pH measurement of urine, for example in a catheter channel or a collection vessel. Oxide layer 610 can be composed of any oxide-based dielectric or insulating material that can be used for insulation in semiconductor devices, including but not limited to silicon dioxide, aluminum oxide, hafnium oxide, and combinations thereof.

The sensing surface 602 can have any shape, including for instance a needle shape. The sensing surface 408 can have a length or diameter of about 5 to about 15 μm, for example from about 5 to about 10 μm or from about 5 to about 8 μm. The sensing surface 602 can be planar or have a three-dimensional shape.

In some embodiments of the invention, the sensing surface 602 includes conducting titanium nitride (TiN). The sensing surface 602 can be composed of any pH sensitive conducting material. In some embodiments of the invention, for example, sensing surface 602 includes a TiN film sputter deposited over a metal line 618. The sensing surface 602 can include, in some embodiments of the invention, platinum, ruthenium oxide, iridium oxide, conductive carbon, or combinations thereof.

In some embodiments of the present invention, a urine pH sensing system includes a plurality of BJT-pH sensors, wherein each BJT-pH sensor includes one sensing surface. In some embodiments of the invention, a surgical apparatus includes a BJT-pH sensor array.

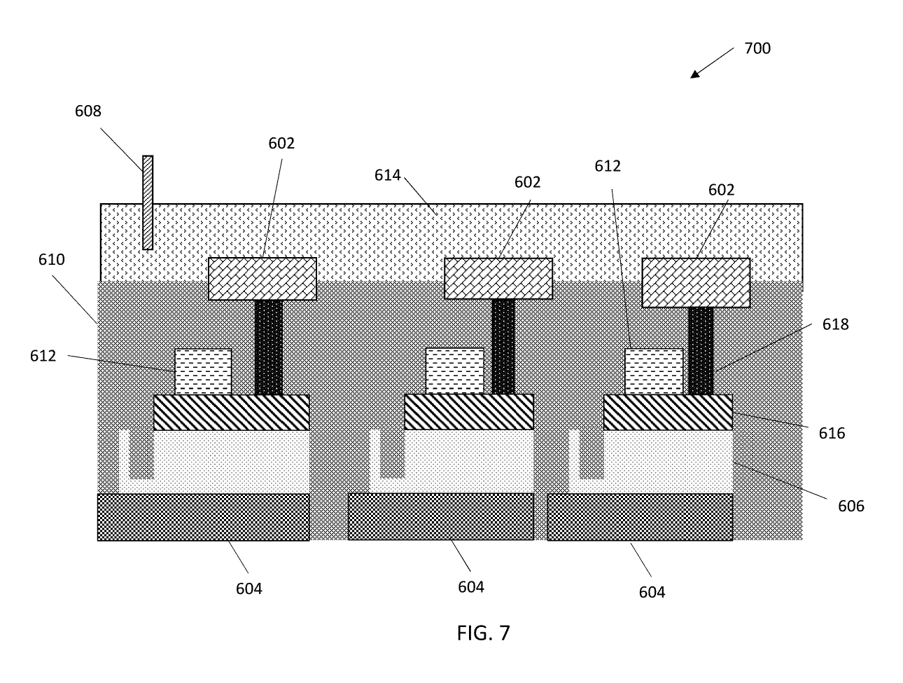

FIG. 7 depicts a cross-sectional side view of a portion of a pH sensor array 700 for use of sensing urine pH according to one or more embodiments of the present invention. The array 700 includes a plurality of sensing surfaces 602. Each of the plurality of sensing surfaces can be connected to a metal line 618. The plurality of sensing surfaces 602 and metal lines 618 can be embedded within an oxide layer 610, such that the sensing surfaces 602 have a surface that can be accessible to a fluid, such as urine in a catheter system. The array 700 includes a reference electrode 608. In some embodiments of the invention, the array 700 includes one reference electrode 608. In some embodiments of the invention, not shown in FIG. 7, the array 700 includes a plurality of reference electrodes 608.

The pH sensor array 700 can include other components, such as each of the components that are included in a BJT-pH sensor 600 according to one or more embodiments of the invention. For example, the plurality of sensing surfaces 602 can each be electrically connected to one or more bases 616 via the plurality of metal lines 618. In some embodiments of the invention, each base 616 is positioned on a collector 606, which is positioned on a substrate 604. In some embodiments of the invention, a pH sensing array 700 includes a plurality of emitters 612.

In operation, in some embodiments of the invention, a pH sensing surface and reference electrode, such as a sensing surface of a BJT-based pH sensor or a FET-based pH sensor, can be brought into contact with urine in a urinary catheter tube or in the collecting vessel of a urinary catheter system. A pH can be determined in real-time and transmitted, via a wired connection or wirelessly, to an external device for reporting and/or recording of pH.

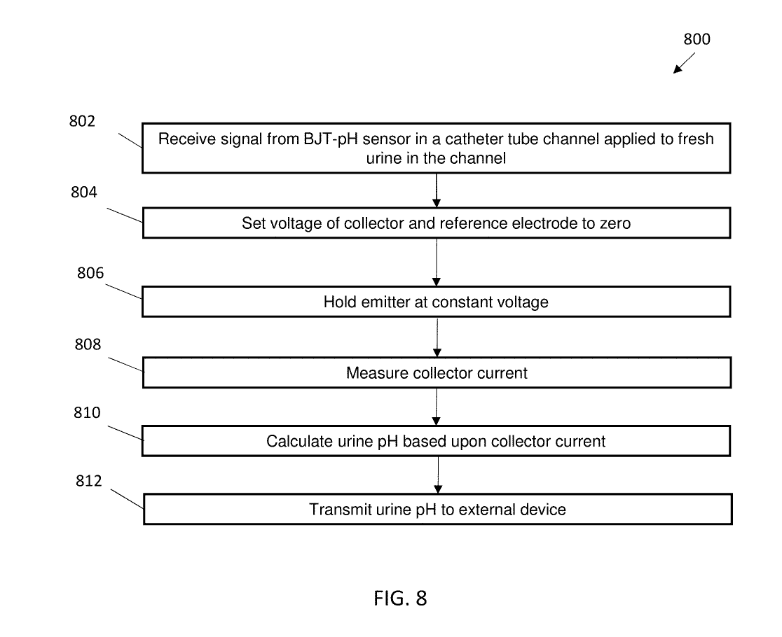

FIG. 8 depicts a flow diagram for an exemplary method 800 of determining urine pH according to one or more embodiments of the present invention. The method 800 includes, as shown at block 802, receiving a signal from a BJT-pH sensor applied to fresh urine. The method 800 also includes, as shown at block 804, setting a voltage of a collector and reference electrode to zero. The method 800 also includes, as shown at block 806, holding an emitter at constant voltage. The method 800 also includes, as shown at block 808, measuring a collector current. The method 800 also includes, as shown at block 810, calculating a urine pH based upon the collector current. The method 800 also includes, as shown at block 812, transmitting the urine pH to an external device.

Embodiments of the present invention can provide a number of technical features and benefits. For example, embodiments of the present invention can provide real-time pH measurements with high sensitivity for detection of a change in a subject's condition. Such measurements can improve the standard of care for subjects by providing early identification of a number of conditions that could benefit from medical intervention, such as kidney malfunction, respiratory problems, impaired perfusion to tissues, or infection. Embodiments of the invention can provide improved care for individuals experiencing one or more conditions resulting in urine pH changes. For instance, a medical professional can obtain accurate and timely notification of pH changes for diabetic patients undergoing treatment.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments of the invention, electronic circuitry including, for example, programmable logic circuitry, field-programmable gate arrays (FPGA), or programmable logic arrays (PLA) may execute the computer readable program instruction by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments described. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments described herein.

1. A system for sensing urine pH, the system comprising:
a urinary catheter tube for insertion into a bladder of a patient, wherein the urine catheter tube comprises an inner cavity;
a collection vessel communicatively coupled to the urinary catheter tube; and
a transistor-based pH sensing device comprising a pH sensing surface and a reference electrode, wherein the pH sensing surface and the reference electrode have a surface exposed to the inner cavity.

2. The system of claim 1, wherein the FET-based pH sensing device further comprises a signal processor.

3. The system of claim 2 further comprising an amplifier in communication with the transistor-based pH sensor and the signal processor.

4. The system of claim 1, wherein the transistor-based pH sensing device further comprises a wireless transmitter.

5. The system of claim 1 further comprising a display.

6. The system of claim 1 further comprising a second transistor-based pH sensing device comprising a second pH sensing surface and a second reference electrode, wherein the second pH sensing surface and the second reference electrode have a surface exposed to the collection vessel.

7. The system of claim 1, wherein the transistor-based pH sensing device comprises a pH sensor array.

8. The system of claim 1, wherein the transistor-based pH sensor is integrated within the urinary catheter tube.

9. The system of claim 1, wherein the transistor-based pH sensor further comprises an oxide layer formed over a source, a drain, and a gate dielectric.

10. The system of claim 9, wherein the pH sensing surface comprises hafnium oxide.

11. The system of claim 1, wherein the transistor-based pH sensor is a FET-based sensor.

12. A system for sensing urine pH, the system comprising
a urinary catheter tube for insertion into a bladder of a patient, wherein the urine catheter tube comprises an inner cavity;
a collection vessel communicatively coupled to the urinary catheter tube; and
a pH sensing device comprising reference electrode and a pH sensing surface connected to the base of a BJT device, wherein the BJT device further comprises a collector and an emitter, and wherein the pH sensing surface and the reference electrode have a surface exposed to the inner cavity.

13. The system of claim 12, wherein the pH sensing device further comprises a signal processor.

14. The system of claim 13 further comprising an amplifier in communication with the pH sensing device and the signal processor.

15. The system of claim 12, wherein the pH sensing device further comprises a wireless transmitter.

16. The system of claim 12 further comprising a display.

17. The system of claim 12 further comprising a second pH sensing device comprising a second pH sensing surface and a second reference electrode, wherein the second pH sensing surface and the second reference electrode have a surface exposed to the collection vessel.

18. The system of claim 12, wherein the pH sensing device comprises a pH sensor array.

19. The system of claim 12, wherein the pH sensing device is integrated within the urinary catheter tube.

20. The system of claim 12, wherein the pH sensing surface comprises TiN.

21. The system of claim 12, wherein the pH sensing device is disposable.

22.-25. (canceled)
